(19) United States
(12) Patent Application Publication
(10) Pub. No.: US 2004/0230205 A1
(43) Pub. Date: Nov. 18, 2004

(54) Device and method to treat genital lesions

(76) Inventor: Syed Rizvi, Bakersfield, CA (US)

Correspondence Address: JEFFREY FURR, 253 N. MAIN STREET, JOHNSTOWN, OH, 43031, US

(21) Appl. No.: 10/249,907

(22) Filed: May 16, 2003

(51) Int. Cl. A61B 17/26

(52) U.S. Cl. 606/110

(57) ABSTRACT

A device and method for diagnosing and treating cervical, vaginal, labial and perineal lesions. Said device comprises a tubular housing having opposing cups on one end that open and close. Cups have grasping, holding and cutting functions. The tubular housing is curved to approximate 45 degrees near the proximal part. Proximal end of the device has a handle, which accommodates three fingers providing ease of operation.

### BACKGROUND OF INVENTION

This invention relates to an implement and methods of use thereof and more particularly to a device to remove the lesions, particularly smaller and pedunculated lesions, from cervix, vagina, labia and perineum for diagnostic and therapeutic purposes. Lesions include but are not limited to Nodules, Polyps, Warts, Cysts, Precancerous and malignant lesions.

Genital warts are growths caused by the human papilloma virus (HPV). Infection of the genital and anal regions with HPV can cause warts on the cervix, vagina, vulva and perineum. Risk factors for genital warts include multiple sexual partners, unknown partners, early onset of sexual activity, tobacco use, nutritional status, hormonal influences, age, stress and concurrent viral infections (such as HIV, Epstein-Barr and herpes). Non-sexual transmission is possible. Diagnosis requires removal of the lesions and histological evaluation. Treatment modalities include excision of the lesion.

Cervical dysplasia also known as cervical intraepithelial neoplasia (CIN) are the precancerous changes of the cervix. The cause is unknown; however, a number of predisposing factors have been identified. Less than 5% of all Pap smear tests conducted note cervical dysplasia. It may occur in women aged 15 and older, with the peak incidence in women aged 25 to 35. Increased risk is associated with: multiple sexual partners, early onset of sexual activity, early childbearing, and past medical history of DES exposure or sexually transmitted diseases, especially genital warts, genital herpes, or HIV infection. The diagnosis requires removal and histological evaluation of the lesion. Cervical dysplasia is a precursor to the cervical cancer. The treatment of cervical dysplasia depends on the degree of dysplasia.

Cervical cancer begins gradually as a pre-cancerous condition called dysplasia. In this form it is easily treatable, usually without the need for hysterectomy. Dysplasia, depending on its severity, can resolve without treatment. But more often it eventually progresses to actual cancer called “carcinoma in situ” (CIS) when it has not yet spread. This process may take many years, but once the cancer is established it quickly spreads further into the nearby tissues or to other organs, usually the intestines, liver, and lung. There are often no symptoms of cervical cancer until the disease is advanced. The overwhelming majority of women diagnosed today with cervical cancer have either not had regular Pap smears or they have not followed up after having an abnormal smear. Not having regular Pap smears is the single greatest risk factor for a bad outcome in women who develop cervical cancer. Removal and histological evaluation of the abnormal areas of the cervix is required for diagnosis. Treatment depends upon the stage of the disease.

Cervical polyps are varying sizes smooth, red or purple fingerlike projections on the cervix. The cause of cervical polyps is not completely understood, but they are frequently the result of infection. They may be associated with chronic inflammation, an abnormal local response to increased levels of estrogen, or local congestion of cervical blood vessels. Cervical polyps are relatively common, especially in women over age 20 who have had children. Only a single polyp is present in most cases but sometimes two or three are found. Removal of the polyp is the typical treatment.

Molluscum contagiosum is caused by a virus that is a member of the Poxvirus family. In adults, molluscum is generally seen on the genitalia as a sexually transmitted disease presenting as single or multiple nodules. Because molluscum produces no systemic illness and is not of long-term public health or economic significance, it has not been treated as other sexually transmitted diseases. The importance is significant in the growing population of immunocompromised patients with AIDS, who may develop a fulminating case of molluscum contagiosum. Treatment modalities, among others, include excision of the lesion

Vaginal cysts are the bulge in the vaginal wall Several types of vaginal cysts exist and vary in sizes. Vaginal inclusion cysts are the most common type of cyst found in the vagina. They may form as a result of trauma sustained by the vaginal walls or after obstetric or gynecologic vaginal procedures, when the vaginal lining doesn't heal to normal smoothness. Gartner's duct cysts develop in the space occupied by the Gartner's duct, usually on the lateral walls of the vagina. This duct is active in the fetus but usually disappears after birth, and portions of the duct may collect fluid and present as a vaginal wall cyst later in life. Benign tumors of the vagina are uncommon and are usually cystic. Treatment options include removal of the cyst.

Vaginal cancer is rare and only makes up about 1% of all gynecologic malignancies. The cause of squamous cell carcinoma of the vagina is unknown, but up to 30% of patients have a prior history of cervical cancer. About 75% of patients with squamous cell cancer of the vagina are over 50. Adenocarcinomas of the vagina more commonly affect younger women. The average age of diagnosis for adenocarcinoma of the vagina is 19. Women whose mothers took diethylstilbestrol (DES) during the first trimester of pregnancy are at increased risk for developing clear cell adenocarcinoma. Sarcoma botryoides of the vagina is a rare type of cancer that mainly occurs in infancy and early childhood. Diagnosis requires excision and histological examination. Treatment depends upon factors like the extend and the type of the disease.

Several instruments in the prior art are known for extracting a biopsy section. A needle instrument for extracting biopsy sections is shown in U.S. Pat. No. 3,606,878. The specimen tissue is drawn by suction into an opening and severed by a slidable cutter. The suction is said to help retain the specimen in the cutter cup.

A double-sided biopsy knife is shown in U.S. Pat. No. 3,327,702 for obtaining biopsies of the cervix. The cutting blades are angled in a manner to provide a conical biopsy. Another biopsy punch is shown in U.S. Pat. No. 2,994,321. This punch utilizes jaws having beaks, which engage and grip a selected tissue portion therebetween. The closing of the jaws frees the tissue specimen from the surrounding tissue.

U.S. Pat. No. 2,778,357 illustrates a biopsy punch having jaw members at one end thereof. Upon closing the jaws, the tissue is captured therebetween. A skin biopsy punch is shown in U.S. Pat. No. 3,515,128 which uses a knife in communication with a piston/cylinder assembly to hold the skin specimen by pressure differential upon such specimen being severed from the attached fatty tissue.

Finally an endocervical strip biopsy instrument is disclosed in U.S. Pat. No. 4,168,698 which uses a basketlike member removably mounted onto a handle. The basket member carries a cutting blade which is drawn distally from the external os of the endocervical canel. During this withdrawal a strip of endocervical tissue falls into the basket.

Although these instruments are assumably effective in their operation, their designs are relatively complex in construction and awkward in use and do not address important issues related to the diagnosis and treatment of female genital lesions. Most of these devices distort the tissue, rendering gross pathological assessment rather difficult if at all possible. These devices also do not address human factors in design and operation. Moreover most of these previous devices have not been particularly designed for disposable use and must be sterilized after removal of the tissue for subsequent use. None of the prior art provides simultaneous and comprehensive diagnostic and therapeutic solutions for lower female genital tract.

Therefore in view of the prior art there is a need for a device and method to solve the issues not addressed by the prior art.

### SUMMARY OF INVENTION

Object of this invention is to provide a device that will allow the operator to perform diagnostic as well as therapeutic procedures on multiple sites with the aid of only one instrument thereby eliminating the need of multiple instruments, procedure steps and treatment sessions.

Another object is to provide a device, which can be used, for procedures on entire lower genital tract including cervix, vagina, labia and perineum. The device can also be used on other parts of the body.

Yet another object is to provide the ability to remove the lesions from cervix, vagina, labia and perineum. Lesions include but are not limited to Nodules, Polyps, Warts, Cysts, Precancerous and malignant lesions.

Still another object is to remove the entire lesion as one specimen.

A further object of the invention is to provide mechanical hemostasis at the operating site.

Yet another object is to provide a disposable multifunction instrument.

Another object is to provide unobstructed view of the lesions and the operating site during the procedure by means of a curve in the shaft of the device.

Another object is to be able to provide the tissue without distortion to aid in pathological diagnosis.

To achieve these and other objectives the present invention provides a device and method aiding in the diagnosis and treatment of cervical, vaginal, labial and perineal lesions. Said device comprises a tubular housing having opposing cups on one end that open and close. Cups have grasping, holding and cutting functions. The tubular housing is curved to approximate 45 degrees near the proximal part of the device. Proximal end of the device has a handle, which accommodates fingers and provides for the operation of the device with ease and consideration of human factors. Tissue removed is sent for histological evaluation as needed. It is to be understood that both the foregoing general description and the following detailed description are exemplary and explanatory but are not restrictive of the invention.

Additional objects and advantages of the invention will be set forth in part in the description which follows, and in part will be obvious from the description, or may be learned by practice of the invention. The objects and advantages are realized and attained by means of the instrumentalities and combinations particularly pointed out in the appended claims.

### BRIEF DESCRIPTION OF DRAWINGS

The accompanying drawing, which is incorporated in and constitutes a part of this specification, illustrates preferred embodiments of the invention and, together with the description, serves to explain the principles of the invention.

FIG. 1. Is a perspective view of the device constructed according to the preferred embodiment of the present invention, with the cups in the fully open position.

Referring now to the drawing in more detail. Numeral 1 designates the distal end of the device with upper and lower cups. Numeral 2 designates the middle portion of the device, which is tubular in shape. Numeral 3 designates the handle of the device.

### DETAILED DESCRIPTION

The device 1 is for the aiding in the diagnosis and treatment of cervical, vaginal, labial and perineal lesions. The device 1, in the preferred emboidment, has an elongated curved tubular housing having distal and proximal ends with a pair of cups 4 on the distal end of the device and a handle on the proximal end of the device 1. Said opposing cups 4 on one end open and close. These cups 4 have grasping, holding and cutting functions. The tubular housing 2 is curved to approximate 45 degrees near the proximal part of the device. Proximal end of the device 1 has a handle 3, which accommodates fingers and provides for the operation of the device with ease and consideration of human factors. Tissue removed is sent for histological evaluation as needed.

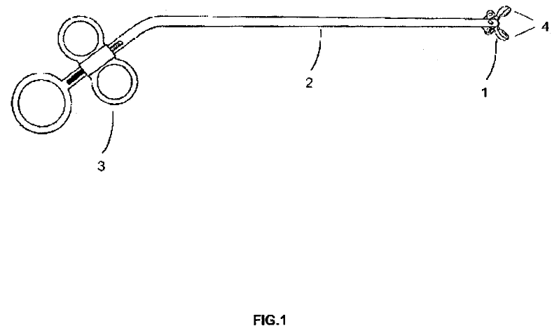

FIG. 1 illustrates a first part of the device 1 in the shape of two opposing cups 4. Diameter of the cups 4 can range from 2 millimeters to 10 millimeters. Cups 4 have deep centers, which allows for encasement of a lesion, protecting it from crushing forces and preventing gross anatomical distortion. Edges of the cups 4 are sharp to provide cutting action if closed completely by force. The cups 4 can be round, triangular or any other shape. If not closed completely the cups 4 perform grasping function and assist in holding and manipulating the lesion.

FIG. 1 also illustrates a second part 2 in the shape of a tubular housing connecting part 1 to part 3. This tubular housing is approximately 8 inches long and approximately 3 millimeter to 4 millimeter wide with or without tapering diameter along the length of the embodiment. This houses the connecting parts between the cups 4 and the handle allowing the cups 4 to open and close. The embodiment is curved at the distal portion to about 45 degrees. This curve aids in providing the unobstructed view of the lesion and the operating site as the operator”s hand is out of the way and is not obstructing the view.

FIG. 1 also illustrates a third part 3 in the shape of a handle with three holes. This entire handle is attached to the distal part of part 2. The dimensions of the part 3 are customary and well known in art. This handle has Upper, lower and middle holes. Upper hole holds the index finger, lower hole holds the middle finger and middle hole holds the thumb. Upper and lower holes of the handle are fixed with the tubular housing (embodiment 2). The middle hole of the handle is movable and is connected to the part in the tubular housing which itself is connected to the cups 4 (part 1). Back and forth motion of the middle hole of the handle opens and closes the cups 4. This type of connection is well known in the art. The upper and lower holes of the handle help stabilize the device and provide grip, motion, force and directional control.

The device 1 can be made of a material that can make the device 1 disposable.

Operation

To remove a polyp surgeon opens the cup 4 by placing index and middle fingers into the upper and lower holes of the handle and pulling the middle hole of the handle backward with the thumb. Now the surgeon places and grasps the polyp in the center of the cup 4. Cups 4 are closed gently by forward movement of the thumb. The base of the polyp is held gently. The entire polyp is twisted and rotated in clockwise direction concurrently increasing the force gradually by forward motion of the thumb. After the polyp is twisted enough to achieve hemostasis, the thumb is moved forward to completely close the cup dissecting the base of the polyp and removing it. To facilitate the rotation, at surgeon”s discretion the fingers can be placed on outer edges of the handle for the duration of rotation.

To remove a wart surgeon opens the cup 4 by placing index and middle fingers into the upper and lower holes of the handle and pulling the middle hole of the handle backward with the thumb. Now the surgeon places and grasps the wart in the center of the cup 4. Cups 4 are closed gently by forward movement of the thumb. The base of the wart is held gently. The entire wart is twisted and rotated in clockwise direction concurrently increasing the force gradually by forward motion of the thumb. After the wart is twisted enough to achieve hemostasis, the thumb is moved forward to completely close the cup dissecting the base of the wart and removing it. To facilitate the rotation, at surgeon”s discretion the fingers can be placed on outer edges of the handle for the duration of rotation.

To remove a cyst surgeon opens the cup 4 by placing index and middle fingers into the upper and lower holes of the handle and pulling the middle hole of the handle backward with the thumb. Now the surgeon places and grasps the cyst in the center of the cup 4. Cups 4 are closed gently by forward movement of the thumb. The base of the cyst is held gently. The entire cyst is twisted and rotated in clockwise direction concurrently increasing the force gradually by forward motion of the thumb. After the cyst is twisted enough to achieve hemostasis, the thumb is moved forward to completely close the cup 4 dissecting the base of the cyst and removing it. To facilitate the rotation, at surgeon”s discretion the fingers can be placed on outer edges of the handle for the duration of rotation.

To remove molluscum contagiosum nodule, surgeon opens the cup 4 by placing index and middle fingers into the upper and lower holes of the handle and pulling the middle hole of the handle backward with the thumb. Now the surgeon grasps the nodule in the center of the cup 4. Cups 4 are closed by forward movement of the thumb dissecting the nodule, which is removed and sent for histological evaluation if so desired.

To remove precancerous or cancerous lesions, surgeon opens the cup 4 by placing index and middle fingers into the upper and lower holes of the handle and pulling the middle hole of the handle backward with the thumb. Now the surgeon grasps the lesion in the center of the cup 4. Cups 4 are closed by forward movement of the thumb dissecting the lesion, which is removed and sent for histological evaluation as needed.

To perform the hemostasis on bleeding site, the edges of the bleeding tissue are grasped between the edges of the cups and held firmly until hemostasis is achieved. This provides for the mechanical occlusion of the bleeding vessels. Other hemostatic aids may also be used in conjunction as needed.

Easy removal of the tissue from the cups 4 is performed by placing the cups in the container with collection fluid and opening the cups 4 by backward movement of the thumb in the middle hole of the handle. Sudden stirring movements easily dislodge the tissue into the fluid.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

It is to be understood that the present invention is not limited to the embodiments described above, but encompasses any and all embodiments within the scope of the claims.

It is to be understood that the foregoing description is exemplary and explanatory but are not restrictive of the invention.

1. A device omprising: An elongated curved tubular housing having distal and proximal ends; a pair of cups on the distal end of the device; and a handle on the proximal end of the device.

2. A device as in claim 1 which is disposable.

3. A device as in claim 1 which allows for unobstructed view of the lesion and the operating site.

4. A device as in claim 1 used for removal of the genital lesions.

5. A device as in claim 1 used to aid in the diagnosis of genital lesions by performing the excision of the lesion for histological evaluation.

6. A device as in claim 1 used to provide a specimen without gross anatomical distortion.

7. A device as in claim 1 used to protect specimen integrity for evaluation.

8. A device as in claim 1 used for hemostasis from bleeding vessels.

9. A device as in claim 1 to used to provide multiple functionalities in one implement.

10. A device as in claim 1 used to remove lesions regardless of the etiology.

11. A device as in claim 1 used to remove an entire lesion in one step.

12. A device as in claim 1 used to aid with the release of removed specimen into collection fluid.

13. A device as in claim 1 used to remove cervical polyps.

14. A device as in claim 1 used to remove cervical polyps with decreased blood loss and without the need of local or general anesthetic.

15. A device as in claim 1 used to remove the genital warts.

16. A device as in claim 1 used to remove genital warts with decreased blood loss and without the need of local or general anesthetic.

17. A device as in claim 1 used to remove cervical cysts

18. A device as in claim 1 used to remove vaginal cysts.

19. A device as in claim 1 used to remove genital warts with decreased blood loss and without the need of local or general anesthetic.

20. A device as in claim 1 used to remove molluscum contagiosum nodule.

21. A device as in claim 1 used to remove material with decreased blood loss and without the need of local or general anesthetic.

22. A method of removing a cervical polyp comprising the steps of: having a device with an elongated curved tubular housing having distal and proximal ends, a pair of cups on the distal end of the device; and a handle on the proximal end with upper and lower holes with a middle hole; placing index and middle fingers into the upper and lower holes of the handle of the said device and pulling the middle hole of the handle backward with the thumb allowing the cups to open; placing and grasping the polyp in the center of the cup; closing the cups gently by forward movement of the thumb; gently holding the polyp at the base; twisting and rotating the polyp in clockwise direction while increasing the force gradually by forward motion of the thumb; closing the cups, after assuring the hemostasis, by moving the thumb forward; and dissecting the base of the polyp and removing it.

23. A method of removing genital warts comprising the steps of: having a device with an elongated curved tubular housing having distal and proximal ends, a pair of cups on the distal end of the device; and a handle on the proximal end with upper and lower holes with a middle hole; placing index and middle fingers into the upper and lower holes of the handle of the said device and pulling the middle hole of the handle backward with the thumb allowing the cups to open; grasping and placing the wart in the center of the cup; closing the cups gently by forward movement of the thumb; gently holding the wart at the base; twisting and rotating the wart in clockwise direction while increasing the force gradually by forward motion of the thumb; closing the cups by moving the thumb forward, after assuring the hemostasis; and dissecting the base of the wart and removing it.

24. A method of removing a cervical cyst comprising the steps of: having a device with an elongated curved tubular housing having distal and proximal ends, a pair of cups on the distal end of the device; and a handle on the proximal end with upper and lower holes with a middle hole; placing index and middle fingers into the upper and lower holes of the handle of the said device and pulling the middle hole of the handle backward with the thumb allowing the cups to open; placing and grasping the cyst in the center of the cup; closing the cups, dissecting the cyst, by forward movement of the thumb; and removing the dissected cyst.

25. A method of removing a vaginal cyst comprising the steps of: having a device with an elongated curved tubular housing having distal and proximal ends, a pair of cups on the distal end of the device; and a handle on the proximal end with upper and lower holes with a middle hole; placing index and middle fingers into the upper and lower holes of the handle of the said device and pulling the middle hole of the handle backward with the thumb allowing the cups to open; placing and grasping the cyst in the center of the cup; closing the cups gently by forward movement of the thumb; gently holding the cyst at the base; twisting and rotating the cyst in clockwise direction while increasing the force gradually by forward motion of the thumb; closing the cups, after assuring the hemostasis, by moving the thumb forward; and dissecting the base of the cyst and removing it.

26. A method of removing a molluscum contagiosum nodule comprising the steps of: having a device with an elongated curved tubular housing having distal and proximal ends, a pair of cups on the distal end of the device; and a handle on the proximal end with upper and lower holes with a middle hole;
placing index and middle fingers into the upper and lower holes of the handle of the said device and pulling the middle hole of the handle backward with the thumb allowing the cups to open; placing and grasping the nodule in the center of the cup; closing the cups by forward movement of the thumb; and dissecting the nodule at the base and removing it.
